(19) United States
(12) Patent Application Publication
(10) Pub. No.: US 2005/0246000 A1
(43) Pub. Date: Nov. 3, 2005

(54) Cooled craniectomy

(75) Inventor: Donald J. Larnard, North Hampton, NH (US)

Correspondence Address: CHRISTOPHER & WEISBERG, P.A., 200 EAST LAS OLAS BOULEVARD, SUITE 2040, FORT LAUDERDALE, FL, 33301, US

(73) Assignee: Seacoast Technologies, Inc.

(21) Appl. No.: 10/837,840

(22) Filed: May 3, 2004

(51) Int. Cl. A61F 7/00; A61F 7/12

(52) U.S. Cl. 607/104; 607/109; 607/113

(57) ABSTRACT

A method and system are provided for reducing the extent and duration of brain swelling in a trauma or stroke victim requiring decompressive craniectomy. In an exemplary method, a medical procedure for inhibiting swelling of the brain includes the steps of performing a decompressive craniectomy to expose intracranial tissue, placing a cooling structure on the exposed intracranial tissue, and cooling the exposed intracranial tissue with the cooling structure. An exemplary system for performing the method includes a source of coolant, a cooling structure, and an umbilical connecting the source of coolant and the cooling structure and conducting cooling fluid therebetween, wherein the cooling structure is dimensioned to cover substantially all exposed intracranial tissue.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a medical method and apparatus for treating swelling of the brain.

### BACKGROUND OF THE INVENTION

In head injuries and stroke, neurological damage generally is caused by swelling of the brain within the confined space of the skull. With increased swelling, the brain can no longer be contained in the cranium and thus shifts, with the brain stem possibly becoming twisted. Blood flow to the brain is impeded resulting in brain damage or death. In trauma cases, further complications can result from a badly contused brain tissue.

The traditional approach to dealing with brain swelling in trauma situations has been to medically manage the patient with various pharmaceuticals, remove brain tissue, or in some cases remove entire lobes of the brain, with certain brain damage resulting. However, in an attempt to spare brain tissue, a procedure has been developed to provide room for the brain to swell. This procedure, known as a decompressive craniectomy, is illustrated in FIG. 1, wherein a relatively large section of the skull 8 of a patient is temporarily removed to immediately relieve pressure on the brain 10. The scalp 12 and the dura mater 14, a tough membrane that covers the brain 10, are then sewn together, and the brain 10 is allowed to continue swelling. Over a period of time that can be as long as 30 days, the brain recedes back into the cranium and the skull is then replaced. During the healing process, patients can wear helmets for protection, and can sometimes carry the portion of the skull that has been removed in their abdomens to allow for the bone marrow to remain alive. Alternatively, the removed skull portion can also be kept outside the body in frozen storage throughout the healing process.

Although the decompressive craniectomy has the potential to save brain tissue, it is believed that patients would further benefit if swelling could be reduced and/or if the duration of the swollen state were reduced.

### SUMMARY OF THE INVENTION

The present invention advantageously provides a method and system for reducing the extent and duration of brain swelling in a trauma or stroke victim, or patient otherwise requiring a decompressive craniectomy.

In an exemplary method, a medical procedure for inhibiting swelling of the brain include the steps of: performing a decompressive craniectomy to expose intracranial tissue and immediately relieve intracranial pressure; placing a cooling structure on the exposed intracranial tissue; and cooling the exposed intracranial tissue with the cooling structure.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates a human skull upon which an example of a prior art decompressive craniectomy procedure has been performed;

FIG. 2 illustrates an exemplary cooling system used to perform a medical procedure in accordance with the present invention;

FIG. 3 illustrates a cooling structure of the system of FIG. 2 positioned within a skull opening after a decompressive craniectomy; and

FIG. 4 illustrates additional details of an exemplary cooling structure.

### DETAILED DESCRIPTION OF THE INVENTION

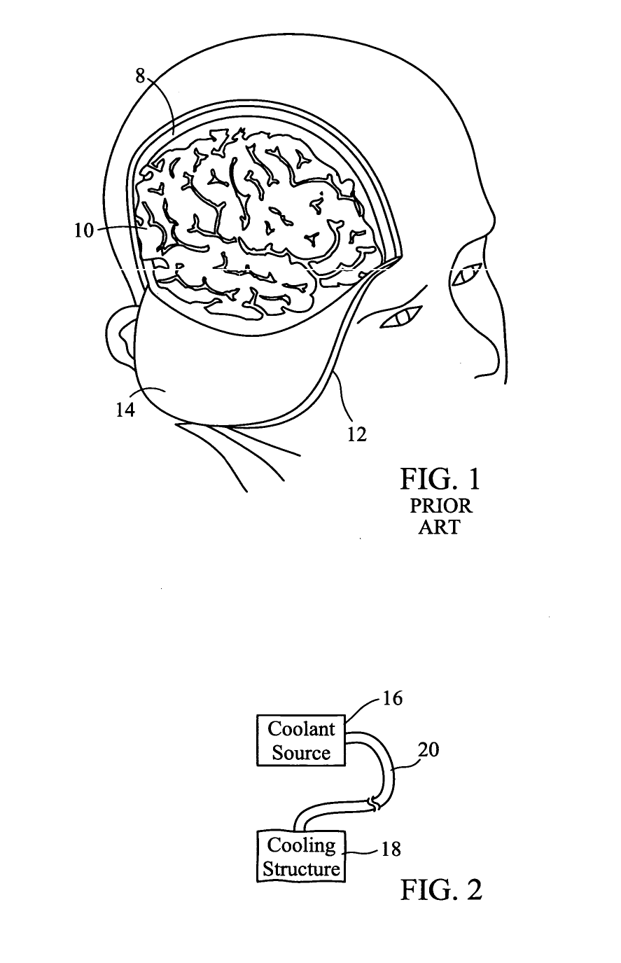

The present invention includes a medical procedure for inhibiting swelling of the brain by locally cooling the brain. In an exemplary procedure, known medical techniques are used to remove a relatively large portion of the skull, such as in the decompressive craniectomy illustrated in FIG. 1. Although a particular location is shown for the cut boundaries, as is a specific size and shape for the cut bone, as used herein, performance of a decompressive craniectomy in intended to encompass any procedure wherein the purpose of the procedure is to allow brain tissue to swell relatively unrestricted and to immediately reduce intracranial pressure by removing at least 2 cm² of bone. Often, much more bone is removed. Again, using known techniques, the dura mater is cut to expose brain tissue or, alternatively, the dura is left intact.

Having determined whether the brain is to be cooled directly or indirectly through the dura, a cooling structure as described below is placed directly on the exposed intracranial tissue. The cooling structure is then cooled to a predetermined temperature below normal body temperature (98.6 degrees F.), but above freezing (32 degrees F.), thereby cooling the intracranial tissue to a temperature above freezing, but below body temperature. The cooling structure is left in place on the intracranial tissues for a selected period of time or until tissue swelling has subsided. To the extent possible, the cooling structure is dimensioned so as to cover and in be in contact with substantially as much of the exposed intracranial tissue as possible.

Turning now to FIG. 2, an exemplary system is illustrated that is suitable for performing the procedure in accordance with the invention. The system includes a source of coolant source 18, a cooling structure 16, and an umbilical 20 connecting the source of coolant and the cooling structure and conducting cooling fluid therebetween. The terms “coolant” and “cooling fluid” as used interchangeably herein generally refer to any flowable substance, including but not limited to gasses, liquids, and combinations thereof. In an exemplary system, the fluid is a saline solution.

Although the source of coolant 18 can be a reservoir from which cooling fluid is expelled to be collected elsewhere or discarded, in view of the expected long duration of the above described treatment, a looped or closed system is believed to be desirable, wherein the cooling fluid is continuously recirculated between the source of coolant and the cooling structure. More particularly, the source of coolant 18 can include a heat exchanger, a pump, and a control mechanism. Temperature, pressure and flow sensors are distributed through the system to provide information to the control mechanism which can, in response to sensor data, alter the performance of the system. The control mechanism can be set or programmed to maintain a specific temperature of the cooling structure and/or to monitor and maintain desired intracranial tissue at a selected temperature.

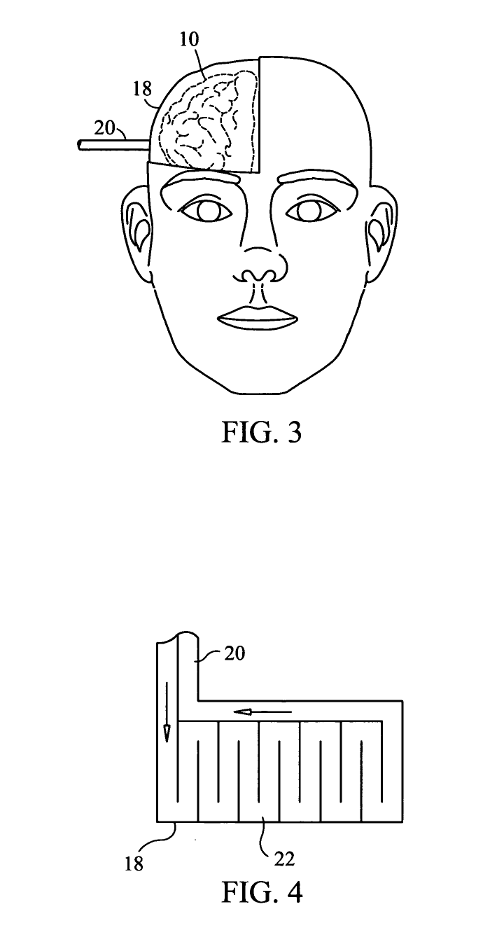

The cooling structure 18 can include a very flexible element, having a surface area greater than two square inches, that readily follows the curvature of the intracranial surface, through which the cooling fluid is circulated. Although the cooling structure 18 can be circular, square, rectangular, or irregular, the particular shape is not significant. Rather, the feature of importance is that the cooling structure 18 covers as much exposed tissue as possible and readily conforms to the tissue without injuring it or causing further trauma as shown in FIG. 3. Exemplary materials suitable for the cooling structure include rubber, silicone, flexible polymers and other bio-compatible materials known in the art. In an exemplary embodiment, as shown in FIG. 4, the cooling structure 18 includes a channel 22 through which cooling fluid travels. The cooling fluid enters and exits the cooling structure 18 through the umbilical 20.

Using a system, such as that described above, a medical procedure is possible for localized cooling of the brain, thereby avoiding systemic temperature reduction throughout the body that can lead to cardiac arrhythmia, immune suppression and coagulopathies.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A medical procedure for inhibiting swelling of the brain comprising the steps of:
performing a decompressive craniectomy to expose intracranial tissue;
placing a cooling structure on the exposed intracranial tissue; and
cooling the exposed intracranial tissue with the cooling structure.

2. The medical procedure of claim 1, wherein the step of placing a cooling structure on the exposed intracranial tissue includes the step of placing the cooling structure over and in contact with substantially all of the exposed intracranial tissue.

3. The medical procedure of claim 2, wherein the step of cooling the exposed intracranial tissue includes the step of cooling a tissue contacting surface of the cooling structure to a temperature in the range of 98 degrees F. to 33 degrees F. for a period of time greater than one day.

4. The medical procedure of claim 3, wherein the period of time greater than one day is a period of time greater than one week.

5. The medical procedure of claim 3, wherein the period of time greater than one day is a period of time greater than one month.

6. The medical procedure of claim 3, wherein the intracranial tissue is brain tissue.

7. The medical procedure of claim 3, wherein the intracranial tissue is dura.

8. A system for inhibiting swelling of the brain following a medical procedure that exposes greater than two square centimeters of intracranial tissue, comprising:
a source of coolant;
a cooling structure dimensioned to cover substantially all exposed intracranial tissue; and
an umbilical connecting the source of coolant and the cooling structure.
